(19) United States
(12) Patent Application Publication
(10) Pub. No.: US 2013/0108713 A1
(43) Pub. Date: May 2, 2013

(54) COMPOSITION POSSESSING ANTIOXIDANT, IMMONOMODULATED, AND ANTITUMOROUS ACTION AND METHOD OF TREATING NEOPLASMS

(71) Applicants: Tkachenko Anatolii Ivanovich, Kharkov (UA); Dacher George Mikhael, Beirut (LB)

(72) Inventors: Tkachenko Anatolii Ivanovich, Kharkov (UA); Dacher George Mikhael, Beirut (LB)

(73) Assignee: RYAOMA, INC., Miami, FL (US)

(21) Appl. No.: 13/563,727

(22) Filed: Jul. 31, 2012

Related U.S. Application Data
(63) Continuation-in-part of application No. 12/492,874, filed on Jun. 26, 2009, now abandoned.

(30) Foreign Application Priority Data
Jun. 26, 2008 (UA) 2008/0853

(51) Int. Cl. A61K 33/18; A61K 31/7034

(52) U.S. Cl. CPC A61K 33/18; A61K 31/7034
USPC 424/670

(57) ABSTRACT

A composition produces antioxidant, immunomodulated, and antitumorous effects. The composition includes tannic acid, potassium iodide, and ascorbic acid. The tannic acid, the potassium iodide; and the ascorbic acid preferably are in a weight ratio of 3:1:1. The potassium iodide is preferably in a mole ratio with the tannic acid of 1:2.5 to 3.5. The composition can be used in methods for treating skin neoplasms by dissolving the composition in a physiological solution and administering the solution intravenously.

### CROSS-REFERENCE TO RELATED APPLICATIONS

This application is a continuation-in-part of prior application Ser. No. 12/492,874, filed Jun. 26, 2009.

This application claims priority under 35 USC §119 of Ukrainian Patent Application No. 2008/08513, which is incorporated by reference.

### STATEMENT REGARDING FEDERALLY SPONSORED RESEARCH OR DEVELOPMENT

Not Applicable

### THE NAMES OF PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The invention relates to compositions and methods for treating immune diseases, in particular, neoplasms of skin, such as melanomas.

2. Description of the Related Art

The search for therapeutically effective compositions in oncology is a top priority issue of present day medical science. In this context, a great interest is taken in both novel synthetic and natural treatment methods and in the composition of well-known preparations.

A malignant skin melanoma is an aggressing type of tumor. From all the types of malignant skin tumors, 65% of lethal outcomes results (in the main) from melanoma. See C. M. Balch C M et al., Cutaneous Melanoma in De Vita VT, CANCER PRINCIPLES AND PRACTICE OF ONCOLOGY 1947-94, (S. Hellman, Jr. & S. A. Rosenberg eds., Lippincott-Raven 5^{th} ed. 1997). The number of melanoma-affected patients is constantly increasing. The survivability median is equal to four to six (4-6) months and is governed by such factors as general state of health and localization of tumors and their number. See W. Tilgen, Malignant Melanoma: Current Therapeutic Concepts, 18 ONCOLOGY 534-47 (1995).

Melanoma can be cured in the early stages of its natural development using surgical approaches. However, with the onset of metastasizing processes melanoma is normally lethal. At this particular stage, the radical surgical treatment is only employed in some rare cases. In contrast, X-ray therapy is utilized solely for a palliative purpose. Therefore, chemotherapy is presently the basic method for melanoma treatment. Chemotherapeutic agents that produce an active impact on melanoma are dacarbazine, platinoid derivatives, different nitrisourea derivatives, and tubulotoxins such as vinblastine and taxans. See M. F. Avril et al. 22 J. CLINICAL ONCOLOGY 1118-25 (2004).

However, the efficiency of the systemic therapy is still rather low with a slight ratio of response in using monoregimes. Polychemotherapy does not excel monochemotherapy in its clinical effects. This is corroborated by the published results from randomized studies ECOG. See P. B. Chapman et al. Phase 3 Multiceriter Randomized Trial of the Dartmouth Regimen Versus Dacarbazine in Patients with Metastatic Melanoma, 17 CLINICAL ONCOLOGY 2745-51 (1999). The present-day methods for treating melanomas do not provide adequate immediate and distant outcomes.

The natural melanoma development assumes a possible modulating role of the host's immune system. From among the modulators of biological response, i.e. cytokins, one can expect the following mechanisms for antitumorous activity: 1) cytostatic effect; 2) stimulation of transferred cell differentiation; 3) antiangiogenic activity; and 4) immunomodulated activity. The latter is associated with induction of molecule expression of the main histocompatibility complex and some tumor-associated antigens. This induction is useful in increasing the identification of tumorous cells of the host immune system.

It is known that indentifying the tumorous cell of the host immune system can hardly be implemented due to linear homopolymer 2-8 bond N-acetylneuraminic acid called polysialic acid (PSA). It is shown that tannic acid enters into reaction with PSA that results in sedimentation of three moles of N-acetylneuraminic acid by each mole of tannic acid. See U.S. Pat. No. 6,063,770 (issued May 16, 2000), IPC 1-7 A61K31/70; A61K31/35. Thus, tannic acid allows the proper immune system of human body to reject the malignant neoplasms just in the same way as an incompatible transplant.

The major component of oncological pathogens is oxidative DNA lesion in which the cell destructors are the active forms of oxygen. See A. Laviano et al. Oxidative Stress and Wasting in Cancer, 10 (4) CURR. OPIN. CLIN. NUTR. METAB. CARE 449-456 (2007). The gallic acid (monomer of tannic acid) is known to be a highly efficient exhibitor of oxygen active form. In the presence of ion-iodide, a redox reaction occurring in a tumor is accompanied by the formation of molecular iodine, which is involved in twinning in galloylated tannic fragments. As the public data suggest, a mixture of tannic acid and potassium iodide in a molar ratio of 1:1 inhibits considerably the formation of free radicals as compared with the same amount of tannic acid itself. See N. S. Khan et al. Anti-oxidant, Pro-oxidant Properties of Tannic Acid and Its Binding to DNA, 125 CHEM. BIOL. INTERACT 177-189 (2000).

However, in spite of the fact that the tannic acid offers an antitumorous antioxidative activity, its high toxicity precludes its application. See S. Tourino et al. Highly Galloylated Tannin Fractions from Witch Hazel (Hamamelis Virginiana) Bark: Electron Transfer Capacity, In Vitro Antioxidant Toxicity, and Effects on Skin-Related Cells, 21(3) CHEM RES TOXICOL. 697-704 (2008).

It is known that ascorbic acid offers a pronounced renovated effect due to the presence of dienole group in its molecule. This promotes regeneration of the normal organism cells destroyed in the course of the tumor development process. D. Ye. Khenson et al. Ascorbic Acid: Biological Functions and the Influence on Oncologic Diseases, 83 (8) THE JOURNAL OF THE G. NUT INSTITUTE OF ONCOLOGIC DISEASES 547-53 (1991).

### BRIEF SUMMARY OF THE INVENTION

It is accordingly an object of the invention to provide a composition possessing antioxidant, immonomodulated, and antitumorous action and a method of treating neoplasms using the composition, which overcome the above-mentioned disadvantages of the heretofore-known compositions and methods of this general type.

With the foregoing and other objects in view there is provided, in accordance with the invention, a compositional remedy possessing antioxidant, immunomodulated, and antitumorous activity.

In accordance with the object of the invention, the combined use of tannic acid and potassium iodide reduces tannic acid's toxic properties. Our experimental data suggest that a joint administration of potassium iodide and tannic acid in a mole ratio 1:2.5-3.5 does not produce any toxic effect.

The problem thus stated can be resolved as follows. Skin tumor affected patients are treated by administering the compositional remedy that comprises tannic acid, potassium iodide, and ascorbic acid in a ratio of weight fractions of 3:1:1. The treatment is performed using the solution of the compositional remedy in physiological solution intravenously, dropwise once per day in a dose of 1.8-2.5 mg/kg of body mass throughout 15 to 20 days.

The technical yield is an increase in efficiency of treating skin tumors through the use of specifically active slightly toxic remedies.

Other features that are considered as characteristic for the invention are set forth in the appended claims.

Although the invention is illustrated and described herein as embodied in a composition possessing antioxidant, immonomodulated, and antitumorous action and a method of treating neoplasms, it is nevertheless not intended to be limited to the details shown, because various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

BRIEF DESCRIPTION OF THE SEVERAL VIEWS OF THE DRAWING

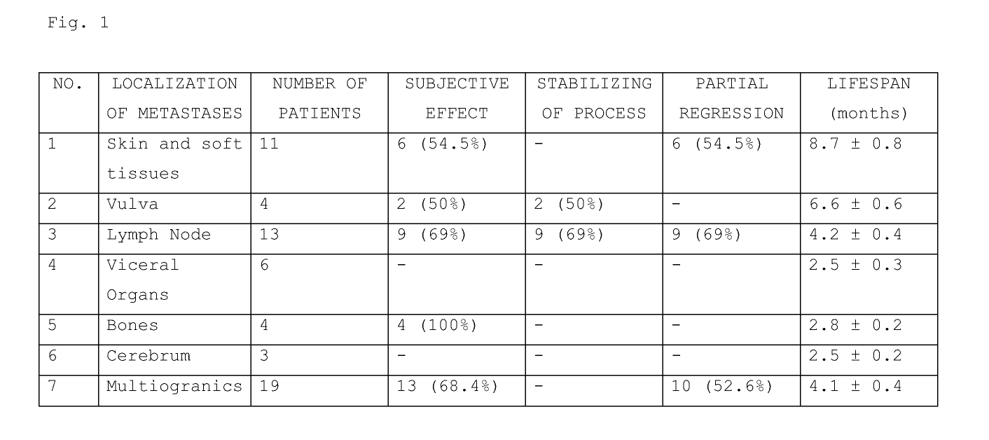

FIG. 1 is a chromatogram of the composition compared to a standard.

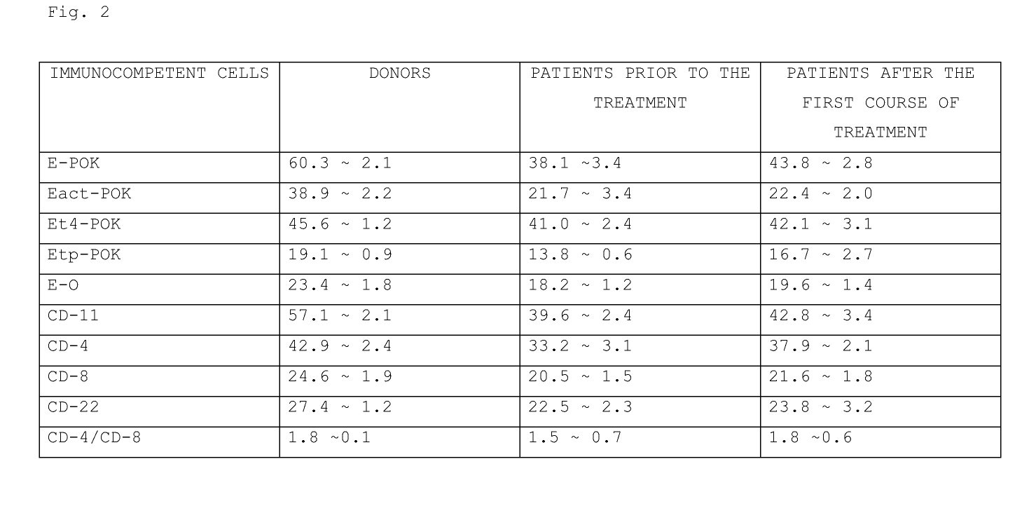

FIG. 2 is a table of results on lifespan of patients given the composition to treat localized metastases.

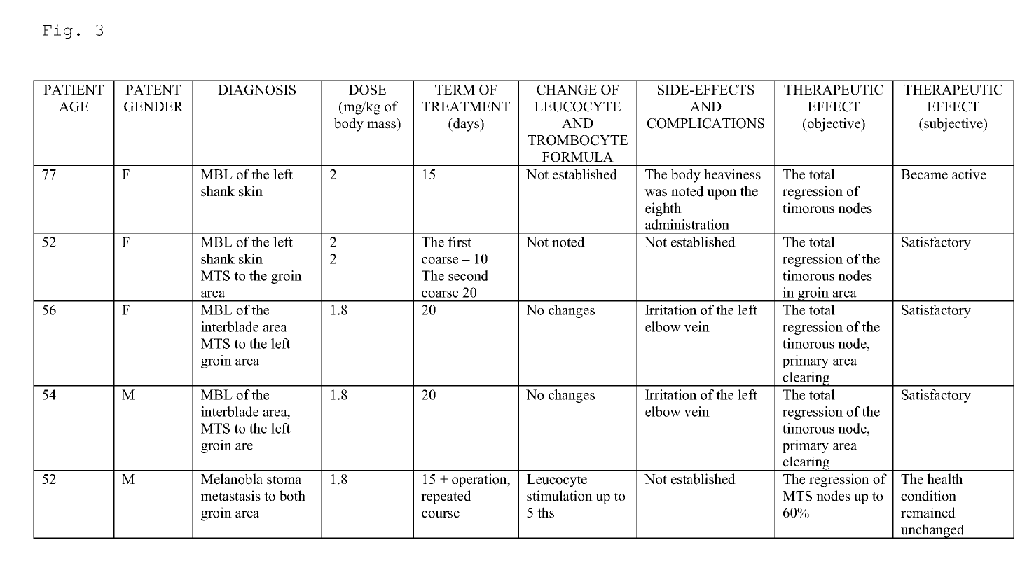

FIG. 3 is a table showing variation in immunocompetent cells content in blood upon treatment with the composition.

### DETAILED DESCRIPTION OF THE INVENTION

The raw materials utilized for manufacturing the composition according to the invention (which is to be sold under the trade name SAMIRIN) are composed of tannic acid containing mainly hydrolyzed tanins—polygalloyl glucose derivatives, which are made by VAG Chermis (Germany). The seria of the tannic acid being used were compared with an operating standard specimen, i.e. tannic acid (CAS Index 1401-55-4) Sigma-Aldrich (USA).

The IR-spectrum of the used tannic acid in KBr tablets contains absorption bands at 3400 cm⁻¹ (valent fluctuations of OH-groups), 1700 cm⁻¹ (valent fluctuations of C═O group of galloyl fragments), 1615 cm⁻¹ (valent fluctuations C═C bonds of aromatic fragments), 1200 cm⁻¹ (C—O—C bonds fluctuations). The general type of the spectra is consistent with the spectra referred to in the literature: R. Gaudreault R et al. Molecular Modeling Poly(Ethylene Oxide) Model Cofactors; 1,3,6-Tri-O-Galloul-Beta-D-Glucose and Coriladgin. 8 J. MOL MODEL 73-80 (2002). The spectrum coincides with the spectrum of the operated standard specimen.

HPLC analysis of tannic acid series has been made using chromato-mass-spectroscopic system, including high-efficiency liquid chromatograph “Agilent 1100 Series” (USA). For the used seria of tannic acid and its operated standard specimen identical chromatograms were obtained. See FIG. 1. In accordance with the data, for the tannic acid, an average molecule mass value of 1700 specific units is accepted. See THE MERCK INDEX, 1615-1616 (13^{th} ed. 2001).

To obtain SAMIRIN, a mixture of tannic acid, potassium iodide, and ascorbic acid is prepared in a ratio of weighting fractions 3:1:1. 180 mg of compositional mixtures are lyophilized, packed into sterile bottles and genetically sealed.

The melanoma-affected patients are treated as follows. SAMIRIN is dissolved in 200 mL of physiological solution and administered intravenously dropwise once per day in a dose of 1.8-2.5 mg/kg of body mass. The treatment course spans 15 to 20 intravenous infusions. When a positive dynamic is achieved, the treatment course is followed until the pronounced effect is produced. The above treatment using SAMIRIN was tested on thirty (30) patients.

In most of clinical studies dacarbazine is a model for comparing between the efficiency and toxicity of novel medications for melanoma treatment. Using dacarbazine in the first chemotherapy line allows one to obtain the objective responses in 11 to 25% of observations with the survivability median between 4.5 to 6 months. See J. K. Luce et al. Dacarbazine-Based Chemotherapy for Metastatic Melanoma: Thirty Year Experience Overview, 19 J. EXP. CLIN. CANCER RES. 21-34 (2000).

A comparative estimate of efficiency and toxicity of SAMIRIN and dacarbazine was held in metastatic melanoma-affected patients including those afflicted by metastases and without them. Along with an estimated rate of objected responses of therapeutical measures, a comparison was made between such indexes as the quality of life, endurability of therapeutic treatment, and total survivability. The study included the patients of the age period between 18-75 with histologically confirmed diagnoses of skin related melanomas. None of the patients had yet gone through chemotherapy. All the patients have given an informed consent to participate in the above investigation. The treatment process continued to the point where the disease started progressing or side-effects developed that required the therapeutic measures to be terminated.

Dacarbazine in a dose of 2500 mg/mL was prescribed in a form of infusion that lasted sixty minutes (60 min.), once a day during successive five days, every four weeks on the 1-5 and 29-33rd days respectively. Repeated courses were carried out only in the patients whose progressing disease was not recorded.

The frequency of objective responses in SAMIRIN-treated patients was 95% and in dacarbazine-treated patients was 23%. The objected responses were recorded in the case of skin-based metastatic melanomas.

During the conducted research dependence of life-span and results of treating for localization of metastases was marked for patients with melanoma during the IV stage of tumor process development. See FIG. 2.

Results shown in the table suggest that administration of the studied medication “SAMARIN” yielded more pronounced effect in the patients with metastases to lymph nodes and multiorgan dissemination. SAMIRIN renders the positive effect on life-span and improves general state of health (diminishes painful symptoms, general weakness, vertigo, promotes a capacity) as compared to patients with dissemination of tumor to visceral organs, bones, and cerebrum.

The most frequent side-effects were related to hematological complications. In SAMIRIN-treated patients, neuropenia was recorded in 0% of observations, and trombocytopenia in 9% of the patients. In Dacarbazine-treated patients, neuropenia was recorded in 14% of observations, and trombocytopenia was recorded in 57% of the patients.

The estimation of SAMIRIN toxic effect suggested that its application results in minimal changes of the patients' state of health that have no influence on his normal vital functions. The SAMIRIN accumulation dynamics in liver allows one to follow up the necessary treatment course in an interrupted mode. The laboratory indications of the patients who received SAMIRIN remain practically unchanged and do not need to be corrected.

Meanwhile, all antitumorous preparations used in chemotherapy exhibit extremely high toxicity. Administering antitumorous preparations results in tumor affected organs being afflicted and also in the pathology-free organs and systems. The immune system is particularly suppressed. See N. L. Perevodchikova, Antitumorous Therapy, PUBL. MEDICINA 166 (Moscow, 1996).

SAMIRIN reveals pronounced immunomodulated effect, which is very important in treating the neglected tumorous stages when a secondary immune deficit arises. See FIG. 3.

The examination of the above-data suggest that all the patients exhibit a pronounced secondary immunodeficit. This is evidenced by a decrease of T-i, in a more slight decrease of β-lymphocytes and their sub-polar make-up. Following the SAMIRIN base-treatment all the patients exhibit a positive dynamic of quantitative indexes of immunocompetent cells as well as the considerable growth in the amounts of lymphocytes-helpers. This process testified to a growth in immunoregulatory index.

However, of the widespread pathologies that give rise to persistent leucopemia and that limit the use of chemotherapy, there is the lesion of lencocyte growth mechanism of blood cells. The studies that have been made suggest that using SAMIRIN remedy does not exercise any influence upon the leukocyte blood formula.

Thus, the elaborated treatment method based on using the SAMIRIN remedy allows one to produce a selective impact upon the tumor growth process and does not have a negative effect on the organs and the systems. The method can be used on all the phases of tumor process development.

The composition can be used to treat hepatitis C, Rheumatoid arthritis, auto-immune diseases, AIDS, and inflammatory bowl disease.

It was discovered that, when tannic acid is combined with potassium iodide, the combination can coagulate with an increased amount of protein.

Preclinical research was conducted on a lyophilized substance containing tannin and potassium iodide. The substance was named “flaraxin”.

The research was performed in the Kiev Scientific Research Institute of Pharmacology and Toxicology. The Institute is part of the Ministry of Health of Ukraine.

The starting point for the research was studies of the tanning properties of the tannin itself. This led to additional research studying the ability of flaraxin (i.e. tannin and potassium iodide) to coagulate protein.

In the coagulation studies, albumin was used for the protein. In a series of experiments, solutions were prepared of albumin, lyophilized flaraxin, and tannin.

The results are described in the table below. In the table, the calculated concentrations of the substances being investigated are listed.

TABLE
CONTENT OF THE ORIGINAL SUBSTANCES AND FLARAXIN IN THE
ANALYTE

| NAME OF SUBSTANCE | TOTAL WEIGHT (g) | TANNIN (g) | KI (g) |
|---|---|---|---|
| Tannin | 0.1987 | 0.1987 | — |
| Flaraxin, lyophilized | 0.2394 | 0.17954 | 0.05984 |
| Flaraxin, lyophilized | 0.2834 | 0.2125 | 0.07085 |
| Flaraxin, lyophilized | 0.2701 | 0.2025 | 006752 |
| Albumin | 0.2069 | — | — |

Note,
the quantity of flaraxin solution taken for the experiments was calculated with regard to the content of tannin in 1 ml of the control solution.
TOTAL WEIGHT = TANIN + KI

### EXPERIMENTAL PROCEDURE

One (1) ml of the tannin solution was put into the test-tube. One (1) ml of the albumin solution was added. 5 ml of water were added. The mixture was shaken. An optic density of the solution was determined on KΦK-2(photoelectric calorimeter) when Δ=260 nm, in cuvette=1 cm in 5, 10, 15, 20, 25 min. The following results were obtained for the optic density:
d₅=0.11
d₁₀=0.16
d₁₅=0.83
d₂₀=0.19
d₂₅=0.19

The data showed that the period of precipitation for the reaction corresponds to 20 min.

Next, to the solution, 0.001 g of potassium iodide was added and its optic density measured: d=0.87. Then, another 0.001 g was added and the optic density measured: d=0.92.

Thus, it was concluded that potassium iodide improved the bonding of the protein with tannin.

Next, the analogical procedures were repeated with different weights of the lyophilized flaraxin. Each weight was repeated three times and the optical density measured. Average results in each case were the following: d2=0.92; d4=1.2.

In addition, the control experiment was conducted with 1 ml of the albumin solution, 0.001 g of potassium iodide, 5 ml of water. It was observed that even one hour later there was no turbidity in the solution.

It was concluded that potassium iodide in considerable quantities induced the coagulation of the albumin solution. The hypothesized mechanism is that coagulation is influenced by the potassium iodide, which acted as an electrolyte in the colloidal solution.

No coagulation between tannin alone was observed.

However, adding small quantities of potassium iodide (1 mg per 5 ml of the solution) to tannin/protein composition resulted in coagulation. In addition, potassium iodide alone did not possess coagulating capacity.

Therefore, it was concluded, tannin in combination with potassium iodide lead to the coagulation of the protein. In addition, changing the amount of potassium iodide relative to the tannin changed the amount of protein being coagulated.

Consequently, the linking capacity of flaraxin with the protein is 4-5 times increased when compared to pure (i.e. without KI) tannin.

1. A composition producing antioxidant, immunomodulated, and antitumorous effects, comprising:
tannic acid;
potassium iodide; and
ascorbic acid;
said tannic acid, said potassium iodide; and said ascorbic acid being in a weight ratio of 3:1:1.

2. A method for treating skin neoplasms, which comprises administering the composition according to claim 1.

3. The method according to claim 2, which further comprises:
dissolving said composition in a physiological solution;
administering said solution intravenously dropwise once per day in a dose of 1.8-2.5 mg/kg of body mass for 15 to 20 days.

4. A composition producing antioxidant, immunomodulated and antitumorous effects, comprising:
potassium iodide; and
tannic acid;
said potassium iodide being in a mole ratio with said tannic acid of 1:2.5-3.5.

5. The method according to claim 1, which further comprises:
dissolving said composition in a physiological solution;
administering said solution intravenously.

6. A method of treating neoplasts, which comprises intravenously administering a composition including tannic acid and potassium iodide.
